(19) United States
(12) Patent Application Publication
(10) Pub. No.: US 2011/0297163 A1
(43) Pub. Date: Dec. 8, 2011

(54) Surgical Procedure Bag

(76) Inventor: Morteza Gharib, Hollidaysburg, PA (US)

(21) Appl. No.: 12/794,761

(22) Filed: Jun. 6, 2010

(51) Int. Cl. A61B 19/08

(52) U.S. Cl. 128/849

(57) ABSTRACT

A surgical procedure bag allowing for rapid preparation of a surgical procedure while ensuring the sterility of the patient and the surgical instruments. The surgical procedure bag comprises a top layer and a bottom layer. The bottom layer is placed above and against the surgical site and, wearing gloves, the surgeon inserts his/her hands through the top layer and manipulates the pre-sterilized surgical instruments placed inside the bag to perform the surgical procedure.

### STATEMENT REGARDING FEDERALLY SPONSORED RESEARCH

Not Applicable.

### FIELD OF INVENTION

This invention relates generally to apparatus used to create a sterile environment during surgical procedures, and more specifically to medical-grade coverings that eliminate the need for a surgeon and his/her assistant(s) to wear a hat, mask or sterile gown.

### BACKGROUND

A surgical procedure is a procedure done on a patient or an animal. It may be a minor procedure such as the closure of a wound or the placement of central line catheter or dialysis catheter. Alternatively, it may be a major operation such as an intra-abdominal or intra-thoracic operation. A major risk in all types of surgical procedures is infection. In order to reduce the risk of infection, surgical procedures are to be performed under strict sterile conditions, which include the preparation of the surgical site with a disinfectant solutions and the covering of the unsterile parts of the patient, the surgeon and the medical staff with sterile coverings.

Typical coverings used by surgeons and medical staff include masks, hats, sterile gowns, and sterile gloves. The donning of all of this apparel is not only time consuming, but there are situations when the surgeon is unable or unwilling to comply with all aseptic requirements due to lack of time in emergency situations or, as happens with many minor surgical procedure or central line placement, due to lack of interest on the part of the surgeon in observing all sterility precautions. With the growing popularity of using ultrasound devices for central venous access procedures, the risk of contaminating a surgical site with an ultrasound probe increases.

Moreover, as is currently done with minor surgical procedures and central line placements outside of hospital operating rooms, sterile packages containing the instruments to be used during the surgical procedure are opened and placed within reach of the surgeon, usually on a table next to the patient. It is not uncommon for the sterilized instruments to then touch unsterile objects or surfaces when they are removed from the packaging to the surgical site.

There is therefore a need in the art for an apparatus that ensures surgical instruments will remain sterilized throughout an entire surgical procedure, thereby reducing the risk of patient infection, while at the same time allowing a surgeon and his/her assistant to quickly prepare for and begin the surgical procedure.

### SUMMARY

The invention relates to an apparatus that ensures surgical instruments will remain sterile throughout an entire surgical procedure, thereby reducing the risk of patient infection, while at the same time allowing a surgeon and his/her assistant to quickly prepare for and begin the surgical procedure. A bag is constructed of transparent, flexible, medical-grade material, such as polyurethane film. The bag comprises a bottom layer and a top layer. The instruments needed to perform the surgical procedure are placed inside the bag and the bag is sterilized and packaged at the manufacturing facility. The bag's bottom layer has an opening, this opening on the periphery of its outside surface, is covered with medical-grade adhesives, such as an acrylic adhesive. This opening will be positioned against the patient's body at the surgical site with the adhesive layer attaching to patient's body and holding the bag in place. The top layer has at least two openings for insertion of the surgeon's hands into the bag. Once the surgeon's hands, covered with sterile gloves, have been inserted into the opening, the surgeon may access the sterilized instruments, and perform the surgical procedure on the patient. The top layer may also include a window covered and sealed with a flap door. This window can be accessed to add or remove surgical instrument throughout surgical procedure. After the surgical procedure is complete, the surgeon withdraws his/her hands from the bag, the bag is removed from the patient, and the bag then disposed of, either with the instruments still inside or after the instruments have been removed from the bag.

The invention can be customized to accomplish a variety of surgical procedures. For major surgeries, larger bags with additional openings for the insertion of one or more assistants' hands or the insertion of extra instruments/equipment may be needed. In one embodiment of the invention customized to provide central venous access in order to place central venous catheters, hemodialysis catheters or pacemakers and so forth, a pouch for insertion of an ultrasound probe is added to the top layer of the bag. This pouch will allow for the placement of the probe directly over the surgical site in order to visualize the vessels underlying the skin while at the same time preventing contamination of the surgical site by the probe. The pouch can be made of any thin flexible, medical-grade material, such as polyurethane film, which is impermeable and allows passage of ultrasound signals from and to the probe. The embodiment with the ultrasound pouch also can be used for regional anesthesia techniques or any other procedure which may require ultrasound visualization of underlying tissues.

### BRIEF DESCRIPTION OF DRAWINGS

FIG. 1 is a perspective view of a surgical bag according to an embodiment.

FIG. 2 is a side sectional view of a surgical bag according to an embodiment.

FIG. 3 is a side sectional view of a surgical bag according to an embodiment.

### DETAILED DESCRIPTION

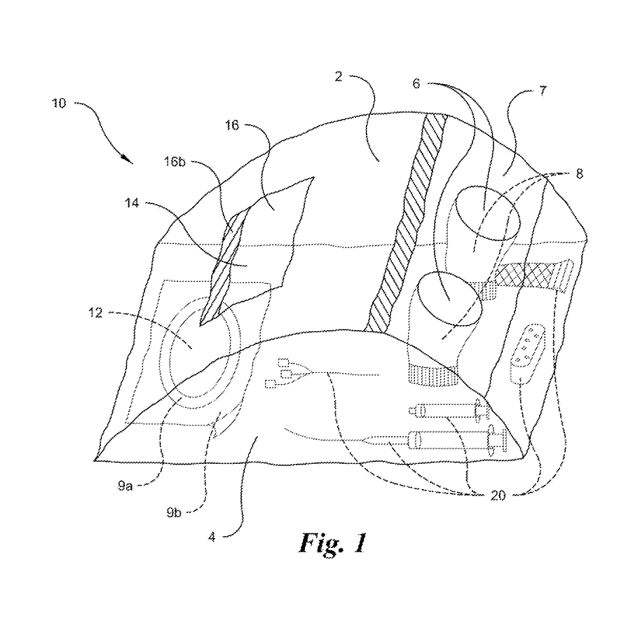

Referring to FIG. 1, a surgical bag 10 according to the present invention is shown. The bag 10 has a bottom layer 4 and top layer 2. The top layer 2 has a plurality of openings 6 that allow for the insertion of a surgeon's hands into the bag 10. The openings 6 may be provided with sleeves 8. The sleeves 8 are sealed to the edges of openings 6 and placed inside the bag 10. The sleeves 8 will cover the surgeon's wrist, and forearm and provides flexibility of hand movement inside the bag 10 while maintaining the sterility of the bag's 10 interior. The sleeves 8 may be constructed out of an elastic and stretchable material, such as polyurethane film. The openings 6 may also have a protective cover 7 that covers the openings 6 from outside of the bag's 10 top layer 2. The protective cover 7 isolates, and protects the sterility of, the interior of bag 10.

The protective cover 7 needs to be released from the top layer 2 and removed from the openings 6 before the surgeon inserts his/her hands into the openings 6. The bottom layer 4 has at least one (1) opening 12. The opening 12 is sized to fit over the surgical site of the patient and to allow the surgeon to access the patient's body so that the surgical procedure may be performed. The opening 12 is provided with a layer of medical grade adhesive 9a on periphery of its exterior surface. The adhesive 9a will attach to the patient's body, at the surgical site, and maintain the bag 10 in place. The opening 12 may be provided with protective cover 9b. Cover 9b will attach to adhesive 9a and cover the opening 12 from outside. Cover 9b needs to be removed before the bag 10 is positioned against the surgical site.

In certain embodiments of the invention, the bag 10 may also comprise an access window 14. The access window 14 is an opening in the top layer 2 that provides access to the interior of the bag 10. The access window 14 is covered with a lid 16. The lid 16 may be resealable against the top layer 2. The lid 16 is comprised of a tab 16b. The tab 16b can be used by an assistant to lift the lid 16. By lifting the lid 16, an assistant can insert additional sterile items into the interior of the bag 10 as needed by surgeon. Used items can be removed from the bag 10 through the access window 14 as well. Care must be taken not to contaminate the interior of the bag 10 when adding items thereto or removing items therefrom through the access window 14.

In certain embodiments, a plurality of surgical instruments 20 are fastened to an interior surface of the bag 10 in releasable manner. Each instrument 20 can be removed from its attachment and put back in place easily. The type of instruments 20 placed inside the bag 10 depends on the type of surgical procedure the bag 10 has been customized to accommodate. The instruments 20 can include needles, syringes, central line catheters, hemodialysis catheters, guide wires, regional anesthesia needles and catheters, scalpels, sharp containers, gauzes, surgical sutures, alcohol preps and other disinfectants, packs of conductive gels, bottles of local anesthetics, or other medications or any other items needed to perform the particular surgical procedure. The bag 10 can be made of any transparent medical grade material or plastic that allows the interior of the bag 10 to be visible, such as polyurethane film.

After the surgical site is prepped in the usual manner, the covering 9b is removed and the bag 10 is positioned on top of the surgical site in such a way that the opening 12 sits directly over and around the surgical site. The cover 7 is then removed and the surgeon's hands, covered with sterile gloves, are placed inside the bag 10 through the openings 6, and the surgical procedure performed in a sterile environment. As can be seen in FIG. 1, because the surgical environment created inside the bag 10 is separated from outside exposure, there should be no need for the surgeon and any assistants to wear a gown, hat or mask.

The present invention has important implications and uses for surgical procedures done in emergency situations or outside of operating rooms, such as surgical procedures done in military zones and so forth where dust from the environment may prevent a sterile procedure from being performed. Even in controlled hospital environments, the isolation from contaminants provided by the present invention will make all medical staff present for the procedure compliant with sterility requirements. Because all the instruments 20 needed for the surgical procedure are organized and accessible inside the bag 10, the surgical procedure can often be done faster and the risk of accidental contamination of the surgical site and the instruments 20 is significantly reduced.

The size of the bag 10 should be customized to suite the surgical procedure intended to be accomplished. As mentioned earlier, the bag 10 can be customized for different surgical procedures and can have multiple openings 6 for the insertion of assistants' hands and other equipment.

The top layer 2 of the bag 10 should be made of transparent medical-grade material, such as polyurethane film. Although the top layer 2 can be made out of material with different rigidity, making it out of soft flexible material has the advantage of having more flexibility for hand movement inside the bag 10. Being soft and collapsible also makes packaging and storing the bag 10 simpler. The bottom layer 4 should be soft and flexible to follow the contour of the patient's body. The bottom 4 should also be impermeable to prevent patient' body fluids from leaking into the bag 10 and contaminating the sterile environment.

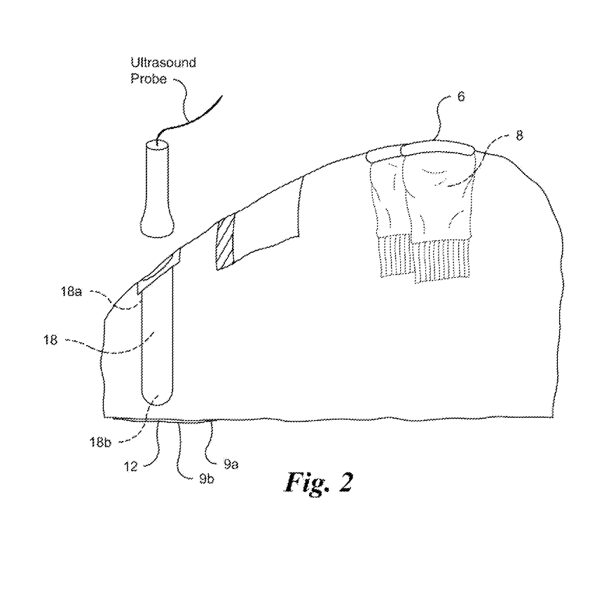

In one embodiment shown in FIG. 2, the bag 10 is comprised of a pouch 18. The pouch 18 is attached to the top layer 2 and is shaped to allow the insertion of an ultrasound probe. The pouch 18 can be made of any thin medical-grade material or plastics that is impermeable to liquids, which allows for easy penetration of an ultrasound signal to and from the ultrasound probe. The pouch 18 is sized to accept a plurality of commercially-available ultrasound probes. The pouch 18 has an open end 18a and a closed end 18b and is attached to the top layer 2 at the open end 18a.

The patient's skin over the target vessel is prepped and the opening 12 of the bag 10 is positioned over the target vessel. The ultrasound probe is then placed inside the pouch 18 and the surgeon places his/her hands, covered with sterile gloves, through the openings 6 and inside the bag 10. Conductive gel may be placed inside the pouch 18 prior to probe insertion. The surgeon can hold the ultrasound probe from inside the bag 10 with one hand to visualize the target vessel without contaminating the surgical field. The other hand is used to perform the endovascular procedure (e.g., advance needles and guide wires into the vessels under direct ultrasound vision). Alternatively, an assistant can hold the ultrasound probe from the outside of the bag 10 without contaminating the patient or the instruments being handled by the surgeon.

In certain embodiments, the pouch 18 can hang free underneath the top layer 2 with the closed end 18b freely moving inside the bag 10. Alternatively, the pouch 18 may be anchored to the bottom layer 4 at the periphery of the opening 12. This anchoring is intended to maintain the head of ultrasound probe over the skin on the target vessel. This anchoring may be rigid, semi-rigid, or even may include flexible anchors or joints to allow the ultrasound probe position and the angle against the patient's skin to be adjustable.

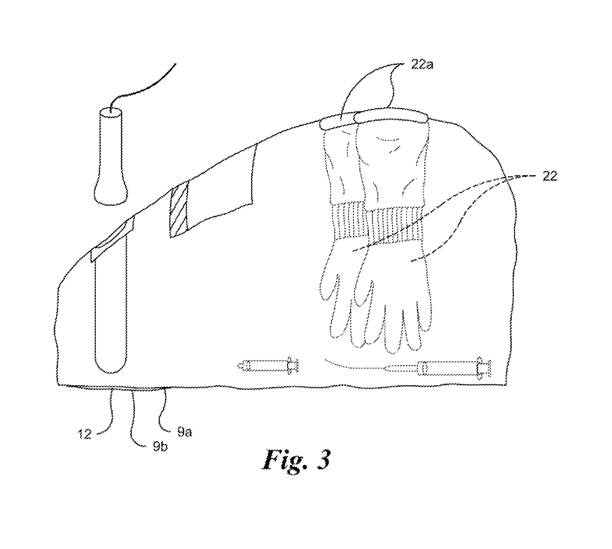

In one embodiment shown in FIG. 3, surgical gloves 22 are placed inside the bag 10. The surgical gloves 22 are attached to top layer 2 and positioned inside the bag 10 as shown in FIG. 3. The proximal ends 22a of the surgical gloves 22 are sealed at the edges of the openings 6. This embodiment can be made with different glove sizes for different hand sizes. One advantage of having surgical gloves 22 incorporated into the bag 10 is that the surgeon can frequently remove his/her hands in and out of the bag 10 without contaminating the interior of the bag 10.

In smaller embodiments customized for minor operations or central vascular access, the bag 10 may be made of a material that, while soft and flexible, allows the bag 10 to naturally maintain its three-dimensional shape to create the space necessary for free movement of the surgeon's hands inside the bag 10. Placement of the hands inside the bag may help to maintain this space. In larger bags, or bags made with very thin walls, means to prevent the bag 10 from collapsing may be needed. These means may include a variety of lightweight skeletons placed inside or outside of the bag 10. The skeleton may be flexible or collapsible to allow easy packaging and storage. Alternatively the bag may be made out of more rigid material or pressurizing the inside of the bag 10 to maintain interior space may be needed in some embodiments. The pressurizing may be accomplished by insufflations of CO₂, air or any other medically approved gas.

I claim:

1. A surgical procedure bag, said bag comprising a top layer and a bottom layer, said top layer comprising a plurality of openings for the insertion of hands into said bag, said bottom layer comprising an opening.

2. The surgical procedure bag of claim 1, wherein said opening of said bottom layer further comprising layer of adhesive covering periphery of said opening over exterior side.

3. The surgical procedure bag of claim 1, wherein said openings of said top layer further comprises a removable protective cover.

4. The surgical procedure bag of claim 1, wherein said opening of said bottom layer further comprises a removable protective cover.

5. The surgical procedure bag of claim 1, wherein said top layer further comprises sleeves attached to said openings.

6. The surgical procedure bag of claim 1, wherein said top layer further comprises a window.

7. The surgical procedure bag of claim 6, wherein said window further comprises a flap door that covers and seals said window.

8. The surgical procedure bag of claim 1, wherein said top layer further comprises a pouch.

9. The surgical procedure bag of claim 8, wherein said pouch is dimensioned to accept commercially available ultrasound probes.

10. The surgical procedure bag of claim 8, wherein said pouch is anchored to said bottom layer of said bag.

11. The surgical procedure bag of claim 1, further comprising surgical gloves attached to said openings of said top layer.

12. The surgical procedure bag of claim 1, wherein said bag is sterile and disposable.

13. The surgical procedure bag of claim 1, wherein said bag is transparent.

14. The surgical procedure bag of claim 1, wherein said bag further comprises means for creating and maintaining space within said bag.

15. A surgical procedure bag, said bag comprising a top layer and a bottom layer, said top layer comprising a plurality of openings for the insertion of hands and a pouch for the insertion of an ultrasound probe, said openings of said top layer further comprising a removable protective cover, said top layer further comprising an access window, said access window comprising a flap door, said bottom layer comprising an opening, said opening further comprising layer of adhesive covering periphery of said opening over exterior side and a removable protective cover.

16. The surgical procedure bag of claim 15, wherein said top layer further comprises sleeves attached to said openings.

17. The surgical procedure bag of claim 15, further comprising surgical gloves attached to said opening of said top layer.

18. The surgical procedure bag of claim 15, wherein said pouch is anchored to said bottom layer of said bag.

19. A method of creating an enclosed sterile environment for performing surgical procedures, said method comprising steps of ;
a) providing a transparent sterile bag, said bag comprising a top layer and bottom layer, said top layer comprising plurality of openings for insertion of hands and surgical instruments, said bottom layer comprising an opening,
b) placing plurality of sterile surgical instruments inside said bag,
c) prepping the surgical site with disinfectant solution,
d) placing said opening of said bottom layer over surgical site,
e) inserting hands covered with sterile surgical gloves through said openings of said top layer into said bag and performing the surgical operation using instruments inside said bag.

20. The method of claim 20, wherein said method further comprising;
a) providing said top layer with a pouch for insertion of ultrasound probe;
b) inserting an ultrasound probe into said pouch and visualizing underlying body tissues.
